(19) United States
(12) Patent Application Publication
(10) Pub. No.: US 2025/0135202 A1
(43) Pub. Date: May 1, 2025

(54) BRAIN STIMULATION SIGNAL CONTROL METHOD, DEVICE AND SYSTEM

(71) Applicant: INDUSTRIAL TECHNOLOGY RESEARCH INSTITUTE, Hsinchu (TW)

(72) Inventor: Chun-Hung LIEN, Taipei City (TW)

(73) Assignee: INDUSTRIAL TECHNOLOGY RESEARCH INSTITUTE, Hsinchu (TW)

(21) Appl. No.: 18/385,206

(22) Filed: Oct. 30, 2023

(51) Int. Cl. A61N 1/36; A61B 5/00

(52) U.S. Cl. CPC A61N 1/36132; A61B 5/4088; A61N 1/36082

(57) ABSTRACT

The present disclosure provides a brain stimulation signal control method, includes: performing a stimulation feedback procedure, including: providing sensory stimulation signals to a user based on a stimulation parameter including a frequency parameter, obtaining a feedback signal in response to the sensory stimulation signals, and determining whether the feedback signal indicates a positive feedback state or a negative feedback state, setting the stimulation parameter as a default parameter when the feedback signal indicates the positive feedback state, and adjusting a value of the stimulation parameter and performing the stimulation feedback procedure again when the feedback signal indicates the negative feedback state.

### BACKGROUND

### 1. Technical Field

This disclosure relates to a brain stimulation signal control method, device and system.

### 2. Related Art

The problem of population aging is becoming increasingly serious, and dementia care also causes heavy economic expenditure. In order to delay or even cure the problem of dementia, various prevention and treatment methods have been developed. For example, current studies have shown that users' cognitive functions can be improved by inducing the brain to produce brain waves in specific frequency bands.

However, due to differences between individuals, a specific frequency band generated by a single stimulus source may not be effective for some users. In addition, in order to confirm whether stimulation is effective, users often need to go to medical institutions for electroencephalogram tests, which is quite inconvenient for users.

### SUMMARY

According to one or more embodiment of this disclosure, a brain stimulation signal control method includes: performing a stimulation feedback procedure, including: providing a plurality of sensory stimulation signals to a user based on a stimulation parameter including a frequency parameter; obtaining a feedback signal in response to the sensory stimulation signals; and determining whether the feedback signal indicates a positive feedback state or a negative feedback state; setting the stimulation parameter as a default parameter when the feedback signal indicates the positive feedback state; and adjusting a value of the stimulation parameter and performing the stimulation feedback procedure again when the feedback signal indicates the negative feedback state.

According to one or more embodiment of this disclosure, a brain stimulation signal control device includes: a plurality of stimulation elements and a control element. The plurality of stimulation elements are configured to output a plurality of sensory stimulation signals to a user based on a stimulation parameter including a frequency parameter. The control element is connected to the plurality of stimulation elements and is configured to control the plurality of stimulation elements to output the plurality of sensory stimulation signals to obtain a feedback signal, wherein the control element sets the stimulation parameter as a default parameter or adjusts the stimulation parameter according to the feedback signal indicating a positive feedback state or a negative feedback state.

According to one or more embodiment of this disclosure, a brain stimulation signal control system includes: a brain stimulation signal control device and a mobile device. The brain stimulation signal control device is configured to output a plurality of sensory stimulation signals to a user based on a stimulation parameter. The mobile device is connected to the brain stimulation signal control device, and configured to perform a stimulation feedback procedure to obtain a feedback signal, and set the stimulation parameter as a default parameter of adjust the stimulation parameter according to the feedback signal indicating a positive feedback state or a negative feedback state.

### BRIEF DESCRIPTION OF THE DRAWINGS

FIG. 1 is a block diagram illustrating a brain stimulation signal control device according to an embodiment of the present disclosure.

FIG. 2 is a block diagram illustrating a brain stimulation signal control system according to an embodiment of the present disclosure.

FIG. 3 is a flowchart illustrating a brain stimulation signal control method according to an embodiment of the present disclosure.

FIG. 4 is a block diagram illustrating a brain stimulation signal control device according to another embodiment of the present disclosure.

FIG. 5 is a block diagram illustrating a mobile device according to an embodiment of the present disclosure.

FIG. 6 is a flowchart illustrating the generation of feedback signal according to an embodiment of the present disclosure.

FIG. 7 is a flowchart illustrating the generation of feedback signal according to another embodiment of the present disclosure.

FIG. 8 is a flowchart illustrating the generation of feedback signal according to yet another embodiment of the present disclosure.

FIG. 9(a) presents the time it takes for a stimulation signal to arrive at the brain using conventional technique; FIG. 9(b) presents the time it takes for a stimulation signal to arrive at the brain using the brain stimulation signal control technique of the present disclosure.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

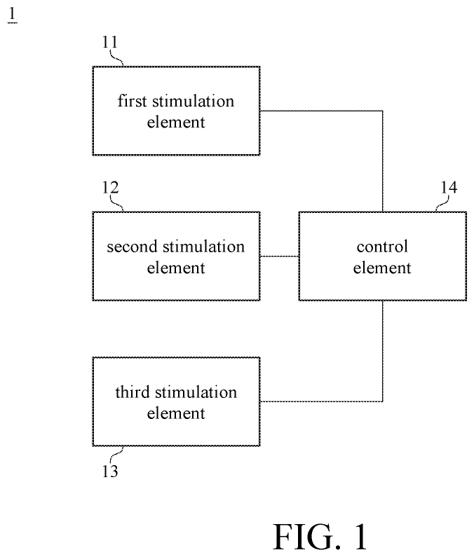

Please refer to FIG. 1, wherein FIG. 1 is a block diagram illustrating a brain stimulation signal control device according to an embodiment of the present disclosure. The brain stimulation signal control device 1 may be implemented as an independent device, such as a head-mounted device, an upright device etc. As shown in FIG. 1, the brain stimulation signal control device 1 includes a first stimulation element 11, a second stimulation element 12, a third stimulation element 13 and a control element 14. The first stimulation element 11, the second stimulation element 12 and the third stimulation element 13 are electrically connected to the control element 14. The first stimulation element 11, the second stimulation element 12 and the third stimulation element 13 are different stimulation elements. For example, the first stimulation element 11 may be a light-emitting diode, the second stimulation element 12 may be a speaker, and the third stimulation element 13 may be a galvanic skin electrode, but the present disclosure is not limited thereto. The control element 14 is configured to control the first stimulation element 11, the second stimulation element 12 and the third stimulation element 13 to output the sensory stimulation signals to user. The control element 14 may include one or more processors, the processor may be, for example, a central processing unit, a graphics processing unit (GPU), a microcontroller, a programmable logic controller or any other processor with signal processing function.

It should be noted that FIG. 1 exemplarily shows three stimulation elements, but the brain stimulation signal control device 1 may only include two of the stimulation elements or include more than three stimulation elements.

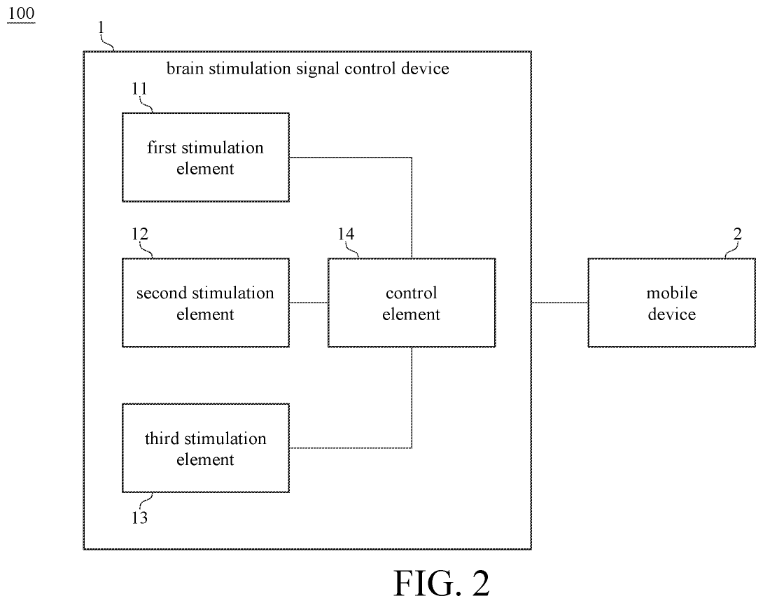

Please refer to FIG. 2, wherein FIG. 2 is a block diagram illustrating a brain stimulation signal control system according to an embodiment of the present disclosure. As shown in FIG. 2, the brain stimulation signal control system 100 includes a brain stimulation signal control device 1 and a mobile device 2. The brain stimulation signal control device 1 of the brain stimulation signal control system 100 may be the same as the brain stimulation signal control device 1 shown in FIG. 1, its descriptions are not repeated herein.

The mobile device 2 may be a smart phone, a tablet and a smart watch etc. The mobile device 2 may be connected to the control element 14 of the brain stimulation signal control device 1 in a wired or wireless way. For example, said wireless way may be wireless communication technique including Bluetooth, Wi-Fi, cellular communication or near field communication etc. The mobile device 2 may be configured to obtain the effect of brain stimulation signals on the user, and control the stimulation parameters of the first stimulation element 11, the second stimulation element 12 and the third stimulation element 13 accordingly.

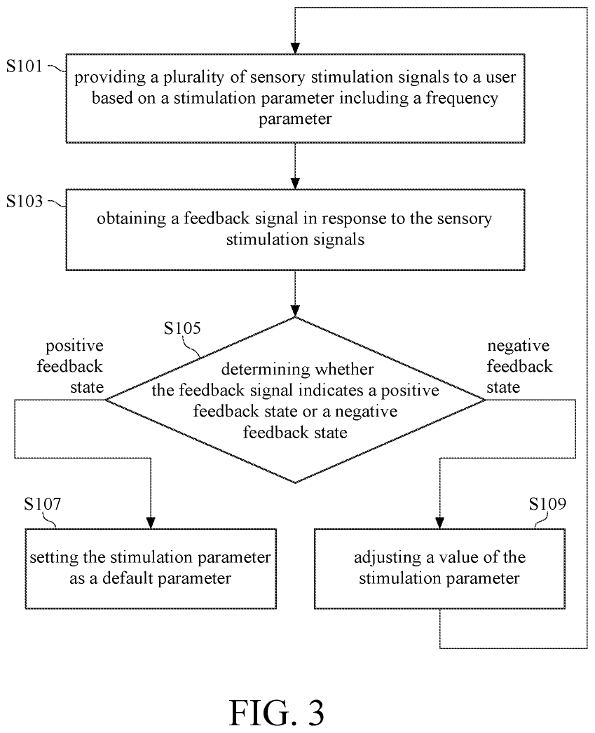

To explain brain stimulation signal control in more detail, please refer to FIG. 2 and FIG. 3, wherein FIG. 3 is a flowchart illustrating a brain stimulation signal control method according to an embodiment of the present disclosure. As shown in FIG. 3, the brain stimulation signal control method includes: step S101: providing a plurality of sensory stimulation signals to a user based on a stimulation parameter including a frequency parameter; step S103: obtaining a feedback signal in response to the sensory stimulation signals; step S105: determining whether the feedback signal indicates a positive feedback state or a negative feedback state; if the determination result of step S105 is “positive feedback state”, performing step S107: setting the stimulation parameter as a default parameter; and if the determination result of step S105 is “negative feedback state”, performing step S109: adjusting a value of the stimulation parameter and performing step S101 again. Steps S101, S103 and S105 may be regarded as a stimulation feedback procedure.

In step S101, the control element 14 controls the first stimulation element 11, the second stimulation element 12 and the third stimulation element 13 to output the sensory stimulation signals respectively to the user based on the stimulation parameter stored by the control element 14. The stimulation parameter includes the frequency parameter, and the sensory stimulation signals may correspond to a same frequency, for example, 40 Hz. Take the above stimulation elements for example, each one of the plurality of sensory stimulation signals is at least one of a light signal, a sound signal and an electrical signal. The sound signal may include a basis sound signal corresponding the frequency parameter and a special sound signal (for example, music, sound of rain, etc.) overlapping the basis sound signal. The light signal may include a basis light signal corresponding the frequency parameter and a special light signal (for example, a video, an image etc.) overlapping the basis light signal. In other words, the control element 14 controls the first stimulation element 11, the second stimulation element 12 and the third stimulation element 13 to output the sensory stimulation signals to the user with the same frequency. In addition, the stimulation parameter may further include at least one of a duty ratio parameter and an intensity parameter, wherein the duty ratio parameter indicates a ratio of a duration of the sensory stimulation signal being at high potential level to a duration of a complete cycle, and the intensity parameter indicates a signal intensity of the sensory stimulation signal.

In the present embodiment, in step S103, the mobile device 2 obtains the feedback signal from the user responding to the sensory stimulation signals. The feedback signal may indicate whether the stimulation parameter corresponding to the sensory stimulation signals matches personalized stimulation needs of the user. For example, the feedback signal may be obtained by detecting user's physiological information (for example, eyeball movement, level of pupil dilation etc.), and the feedback signal may also include user's subjective feedback (for example, obtained by using a questionnaire) etc.

In step S105, the mobile device 2 determines whether the feedback signal indicates the positive feedback state or the negative feedback state. When the feedback signal indicates the positive feedback state, it means that the stimulation parameter of the sensory stimulation signals output to the user in step S101 matches the personalized stimulation need of the user. When the feedback signal indicates the negative feedback state, it means that the stimulation parameter of the sensory stimulation signals output to the user in step S101 does not match the personalized stimulation need of the user.

Therefore, when the feedback signal indicates the positive feedback state, in step S107, the mobile device 2 sets the stimulation parameter of the sensory stimulation signals output in step S101 as the default parameter (meaning, set the stimulation parameter as a standard formula), and store the default parameter into the control element 14. Therefore, when the brain stimulation signal control device is activated again later on, the control element 14 may use the default parameter that matches the personalized stimulation need of the user to control the first stimulation element 11, the second stimulation element 12 and the third stimulation element 13 to output the sensory stimulation signals to the user.

On the contrary, when the feedback signal indicates the negative feedback state, in step S109, the mobile device 2 adjusts the value of the stimulation parameter, and performs the stimulation feedback procedure again. Take the frequency parameter for example, the mobile device 2 may adjust the stimulation parameter within a default range, and use the adjusted stimulation parameter to perform step S101 again. A default frequency range is preferably a frequency range that is medically proven to be suitable for most users, such as a range between 38 Hz and 42 Hz. In addition, the duty ratio parameter and the intensity parameter may have a default duty ratio range and a default control degree, respectively. The mobile device 2 may adjust the stimulation parameter accordingly. The default duty ratio range is, for example, a range between 25% and 35%, and the default control degree of the intensity parameter is, for example, 5%, but the present disclosure is not limited thereto. The default duty ratio range and the default control degree may be adjusted for different scenarios.

The above uses the brain stimulation signal control system 100 of FIG. 2 to explain the steps shown in FIG. 3, but in an embodiment, the brain stimulation signal control device 1 of FIG. 1 may be also used to perform the steps shown in FIG. 3. Specifically, in step S101, the control element 14 controls the first stimulation element 11, the second stimulation element 12 and the third stimulation element 13 to output the sensory stimulation signals to the user based on the pre-stored stimulation parameter. In step S103, the control element 14 may obtain the feedback signal from the mobile device 2 of the user, or may obtain the feedback signal directly from the user in response to the sensory stimulation signals. In step S105, the control element 14 determines whether the feedback signal indicates the positive feedback state or the negative feedback state. When the feedback signal indicates the positive feedback state, in step S107, the control element 14 sets the stimulation parameter as the default parameter (meaning, set the stimulation parameter as a standard formula), and stores the default parameter. When the feedback signal indicates the negative feedback state, in step S109, the control element 14 adjusts the stimulation parameter within the default frequency range, and performs step S101 again using the adjusted stimulation parameter.

Through the operation of the brain stimulation signal control device 1 and the brain stimulation signal control system 100 adjusting the stimulation parameter of the sensory stimulation signals according to the feedback signal, a personalized stimulation parameter may be obtained. The brain stimulation signal control device 1 and the brain stimulation signal control system 100 may use the personalized stimulation parameter of the user to provide the corresponding sensory stimulation signals to the user, thereby improving the effectiveness of improving the user's cognitive function.

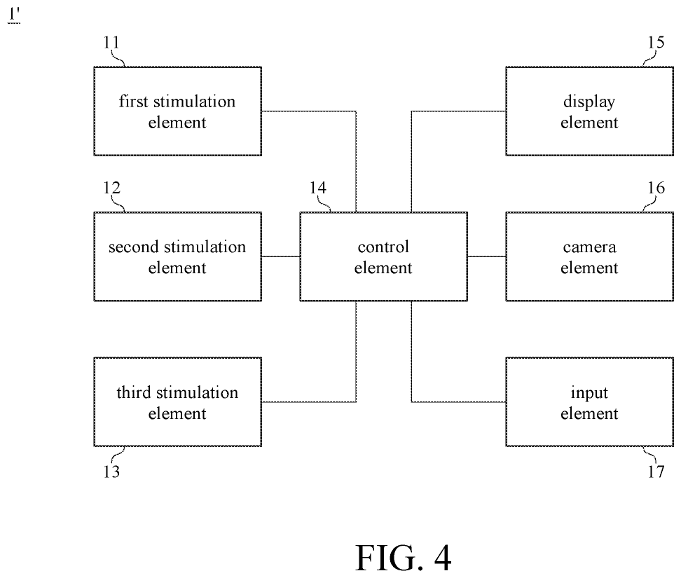

Please refer to FIG. 4, wherein FIG. 4 is a block diagram illustrating a brain stimulation signal control device according to another embodiment of the present disclosure. As shown in FIG. 4, the brain stimulation signal control device l′ includes a first stimulation element 11, a second stimulation element 12, a third stimulation element 13, a control element 14, a display element 15, a camera element 16 and an input element 17. The first stimulation element 11, the second stimulation element 12, the third stimulation element 13 and the control element 14 of the present embodiment may be the same as the first stimulation element 11, the second stimulation element 12, the third stimulation element 13 and the control element 14 shown in FIG. 1, respectively, and their descriptions are not repeated herein.

The display element 15, the camera element 16 and the input element 17 may be electrically connected to the control element 14. The display element 15 may be configured to display images, videos and/or texts. The camera element 16 may be configured to photograph the user. The input element 17 may be configured to receive a command input by the user, wherein the input element 17 may be a mouse, a keyboard or a microphone etc. In other words, the display element 15, the camera element 16 and the input element 17 may all be used to receive the user's feedback to generate the feedback signal. Further, in an embodiment, the display element 15 may be a touch screen, and the input element 17 may be omitted. Or, in another embodiment, the display element 15 and the input element 17 may be kept and the camera element 16 may be omitted. That is, the display element 15, the camera element 16 and the input element 17 are elements selectively disposed, and may be disposed according to requirements in different embodiments, and is not limited herein.

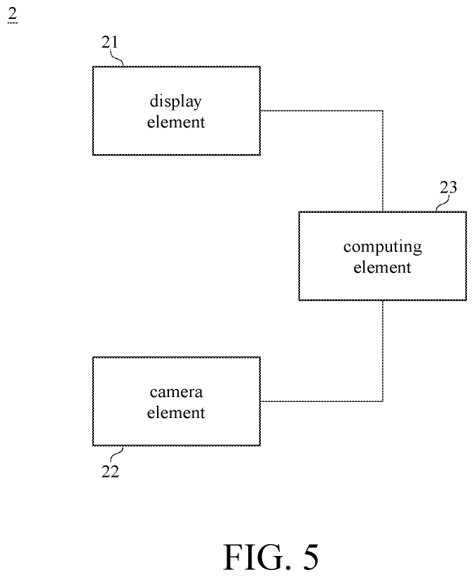

Please refer to FIG. 5, wherein FIG. 5 is a block diagram illustrating a mobile device according to an embodiment of the present disclosure. As shown in FIG. 5, the mobile device 2 may include a display element 21, a camera element 22 and a computing element 23. The computing element 23 is electrically connected to the display element 21 and the camera element 22, and the computing element 23 may be further electrically connected to the control element 14 shown in FIG. 1 or FIG. 2. The display element 21 may be the same as the display element 15 shown in FIG. 4, and may be configured to display images, videos and/or texts. The display element 21 may be a touch screen. The camera element 22 may be the same as the camera element 16 shown in FIG. 4, and may be disposed at the same side as the display element 21 to photograph the user's pupil. In other words, the display element 21, the camera element 22 and the computing element 23 may all be configured to receive user's feedback to generate the feedback signal.

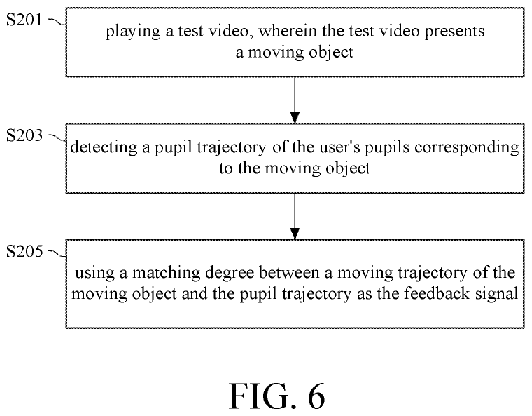

Please refer to FIG. 5 and FIG. 6, wherein FIG. 6 is a flowchart illustrating the generation of feedback signal according to an embodiment of the present disclosure. FIG. 6 may be regarded as a detailed flowchart of an embodiment of step S103 of FIG. 3. As shown in FIG. 6, obtaining the feedback signal of the user includes: step S201: playing a test video, wherein the test video presents a moving object; step S203: detecting a pupil trajectory of the user's pupils corresponding to the moving object; and step S205: using a matching degree between a moving trajectory of the moving object and the pupil trajectory as the feedback signal.

In step S201, the display element 21 plays the test video, wherein the test video shows the moving object (for example, a dot, a pattern etc.) moving in the picture. In step S203, the camera element 22 detects the tracking performed by the user's pupils on the moving object to obtain the pupil trajectory. In step S205, the computing element 23 uses the matching degree between the moving trajectory of the moving object and the pupil trajectory as the feedback signal, wherein the positive feedback state indicates that the matching degree is equal to or higher than the default degree, and the negative feedback state indicates that the matching degree is lower than the default degree. The matching degree may include an overlapping degree between the moving trajectory and the pupil trajectory, or a time difference between one or more points on the moving trajectory and the corresponding one or more points on the pupil trajectory. In other words, the higher the overlapping degree is, the higher matching degree is. The higher the time difference is, the lower matching degree is.

For example, when the matching degree between the moving trajectory and the pupil trajectory is equal to or higher than the default degree, it means that the stimulation parameter of the sensory stimulation signals output to the user matches the personalized stimulation need of the user, so that user's eye-tracking ability matches an expected state. Therefore, the feedback signal indicates the positive feedback state.

On the contrary, when the matching degree between the moving trajectory and the pupil trajectory is lower than the default degree, it means that the stimulation parameter of the sensory stimulation signals output to the user does not match the personalized stimulation need of the user, and does not allow user's eye-tracking ability to match the expected state. Therefore, the feedback signal indicates the negative feedback state.

The brain stimulation signal control device 1′ shown in FIG. 4 is also adapted to steps shown in FIG. 6. That is, the display element 15 of FIG. 4 may perform step S201, the camera element 16 may perform step S203, and the control element 14 may perform step S205, the details of performing the steps are described above, their descriptions are not repeated herein.

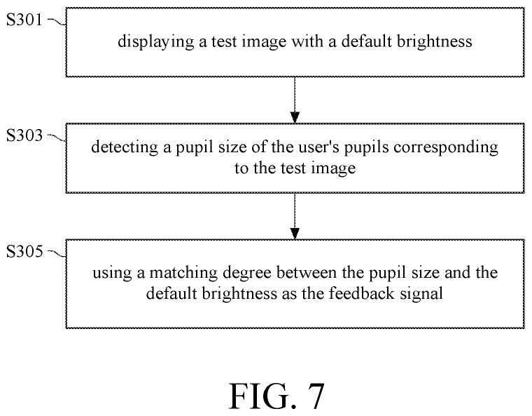

Please refer to FIG. 5 and FIG. 7, wherein FIG. 7 is a flowchart illustrating the generation of feedback signal according to another embodiment of the present disclosure. FIG. 7 may be regarded as a detailed flowchart of an embodiment of step S103 of FIG. 3. As shown in FIG. 7, obtaining the feedback signal of the user includes: step S301: displaying a test image with a default brightness; step S303: detecting a pupil size of the user's pupils corresponding to the test image; and step S305: using a matching degree between the pupil size and the default brightness as the feedback signal.

In step S301, the display element 21 displays the test image in the default brightness, the present disclosure does not limit the specific content of the test image. In step S303, the camera element 22 photographs user's pupils to obtain the pupil size corresponding to when the user is viewing the test image. In step S305, the computing element 23 uses the matching degree between the default brightness and the pupil size as the feedback signal, wherein the positive feedback state indicates that the matching degree is equal to or higher than the default degree, and the negative feedback state indicates that the matching degree is lower than the default degree. For example, the default brightness may have a corresponding default pupil size (for example, the higher the default brightness is, the smaller the corresponding default pupil size is), the closer the pupil size is to the default pupil size, the higher the matching degree is; the further away the pupil size is from the default pupil size, the lower the matching degree is.

The brain stimulation signal control device 1′ shown in FIG. 4 is also adapted to steps of FIG. 7. That is, the display element 15 of FIG. 4 may perform step S301, the camera element 16 may perform step S303, and the control element 14 may perform step S305, the details of performing the steps are described above, their descriptions are not repeated herein.

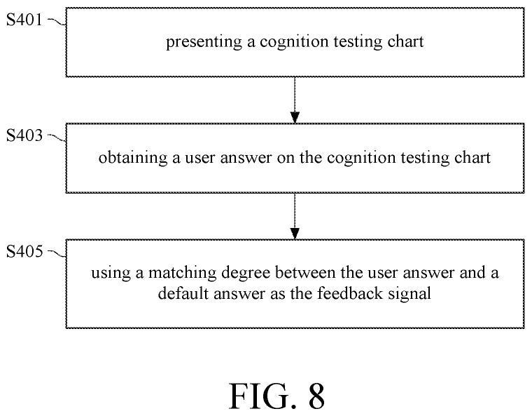

Please refer to FIG. 5 and FIG. 8, wherein FIG. 8 is a flowchart illustrating the generation of feedback signal according to yet another embodiment of the present disclosure. FIG. 8 may be regarded as a detailed flowchart of an embodiment of step S103 of FIG. 3. As shown in FIG. 8, obtaining the feedback signal of the user includes: step S401: presenting a cognition testing chart; step S403: obtaining a user answer on the cognition testing chart; and step S405: using a matching degree between the user answer and a default answer as the feedback signal.

In step S401, the display element 21 presents the cognition testing chart. The cognition testing chart may include one or more questions for testing one or more of memory ability, perceptual movement, execution ability and concentration ability. In step S403, the mobile device 2 obtains the answers to the questions provided by the user as the user answer. For example, the user answer may be input through the display element 21 (where the display element 21 is a touch screen) or through a microphone of the mobile device 2. In step S405, the computing element 23 compares the user answer with the default answer to determine the matching degree, wherein the positive feedback state indicates that the matching degree is equal to or higher than the default degree, and the negative feedback state indicates that the matching degree is lower than the default degree. The matching degree may be a degree of similarity between the user answer and the default answer (i.e. the accuracy rate). In other words, the higher the accuracy rate of the user answer is, the higher the matching degree is.

The brain stimulation signal control device l′ shown in FIG. 4 is also adapted to steps in FIG. 8. That is, the display element 15 in FIG. 4 may perform step S401, the input element 17 may perform step S403, and the control element 14 may perform step S405, the details of performing the steps are described above, their descriptions are not repeated herein.

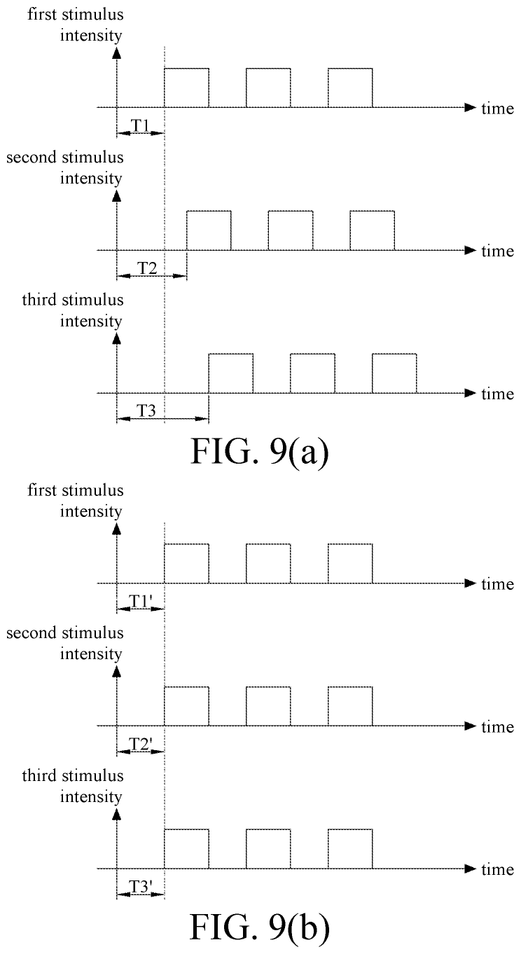

Please refer to FIG. 9(a) and FIG. 9(b), wherein FIG. 9(a) presents the time it takes for a stimulation signal to arrive at a target area (for example, brain) of the user using conventional technique; and FIG. 9(b) presents the time it takes for a stimulation signal to arrive at a target area (for example, brain) of the user using the brain stimulation signal control technique of the present disclosure. In FIG. 9(b), the first stimulus intensity may be the intensity of the sensory stimulation signal output by the first stimulation element 11 described above, the second stimulus intensity may be the intensity of the sensory stimulation signal output by the second stimulation element 12 described above, and the third stimulus intensity may be the intensity of the sensory stimulation signal output by the third stimulation element 13 described above. Further, in the present embodiment, the first stimulation element 11, the second stimulation element 12 and the third stimulation element 13 output the sensory stimulation signals with the frequency parameter of 40 Hz.

Please refer to FIG. 9(a) first. Taking the brain as the target area as an example, in the conventional technique, time required for the sensory stimulation signal output by the first stimulation element to arrive at the brain of the user is a first duration T1, time required for the sensory stimulation signal output by the second stimulation element to arrive at the brain of the user is a second duration T2, and time required for the sensory stimulation signal output by the third stimulation element to arrive at the brain of the user is a third duration T3. As shown in FIG. 9(a), the first duration T1, the second duration T2 and the third duration T3 have time differences therebetween (for example, a time difference between two adjacent sensory stimulation signals is 25 ms), it means that the sensory stimulation signals do not arrive at the brain area of the user at the same time by using the conventional technique.

Please refer to FIG. 9(b). Taking the brain as the target area as an example, time required for the sensory stimulation signal output by the first stimulation element 11 to arrive at the brain of the user is a first duration first duration T1′, time required for the sensory stimulation signal output by the second stimulation element 12 to arrive at the brain of the user is a second duration T2′, and time required for the sensory stimulation signal output by the third stimulation element 13 to arrive at the brain of the user is a third duration T3′. As shown in FIG. 9(b), the first duration T1′, the second duration T2′ and the third duration T3′ do not have time difference therebetween, it means that the sensory stimulation signals may arrive at the brain area of the user almost at the same time by using the brain stimulation technique of the present disclosure.

In view of the above description, the brain stimulation signal control method, device and system according to one or more embodiments of the present disclosure may allow the stimulation parameter of the sensory stimulation signals to be more personalized by adjusting the stimulation parameter according to the feedback signal of the user. In addition, by obtaining the feedback signal of the user with the mobile device and using the feedback signal as the basis for adjusting the stimulation parameter of the sensory stimulation signals, the sensory stimulation signals may be adjusted without the user traveling to medical institutions, allowing brain stimulation to be easily integrated into daily use scenarios.

What is claimed is:

1. A brain stimulation signal control method, comprising:
performing a stimulation feedback procedure, including:
providing a plurality of sensory stimulation signals to a user based on a stimulation parameter including a frequency parameter;
obtaining a feedback signal in response to the plurality of sensory stimulation signals; and
determining whether the feedback signal indicates a positive feedback state or a negative feedback state;
setting the stimulation parameter as a default parameter when the feedback signal indicates the positive feedback state; and
adjusting a value of the stimulation parameter and performing the stimulation feedback procedure again when the feedback signal indicates the negative feedback state.

2. The brain stimulation signal control method according to claim 1, wherein obtaining the feedback signal in response to the plurality of sensory stimulation signals comprises:
playing a test video, wherein the test video presents a moving object;
detecting a pupil trajectory of the user's pupils corresponding to the moving object; and
using a matching degree between a moving trajectory of the moving object and the pupil trajectory as the feedback signal,
wherein the positive feedback state indicates the matching degree being higher than or equal to a default degree, and the negative feedback state indicates the matching degree being lower than the default degree.

3. The brain stimulation signal control method according to claim 1, wherein obtaining the feedback signal in response to the plurality of sensory stimulation signals comprises:
displaying a test image with a default brightness;
detecting a pupil size of the user's pupils corresponding to the test image; and
using a matching degree between the pupil size and the default brightness as the feedback signal,
wherein the positive feedback state indicates the matching degree being higher than or equal to a default degree, and the negative feedback state indicates the matching degree being lower than the default degree.

4. The brain stimulation signal control method according to claim 1, wherein obtaining the feedback signal in response to the plurality of sensory stimulation signals comprises:
presenting a cognition testing chart;
obtaining a user answer on the cognition testing chart; and
using a matching degree between the user answer and a default answer as the feedback signal,
wherein the positive feedback state indicates the matching degree being higher than or equal to a default degree, and the negative feedback state indicates the matching degree being lower than the default degree.

5. The brain stimulation signal control method according to claim 1, wherein the plurality of sensory stimulation signals are capable of arriving at a target area of the user substantially simultaneously, and the plurality of sensory stimulation signals correspond to a same frequency.

6. The brain stimulation signal control method according to claim 1, wherein each one of the plurality of sensory stimulation signals is at least one of a light signal, a sound signal and an electrical signal.

7. The brain stimulation signal control method according to claim 1, wherein the stimulation parameter further comprises at least one of a duty ratio parameter and an intensity parameter.

8. A brain stimulation signal control device, comprising:
a plurality of stimulation elements configured to output a plurality of sensory stimulation signals to a user based on a stimulation parameter; and
a control element connected to the plurality of stimulation elements and configured to control the plurality of stimulation elements to output the plurality of sensory stimulation signals to obtain a feedback signal,
wherein the control element sets the stimulation parameter as a default parameter or adjusts the stimulation parameter according to the feedback signal indicating a positive feedback state or a negative feedback state.

9. The brain stimulation signal control device according to claim 8, further comprising:
a display element connected to the control element and configured to play a test video, wherein the test video presents a moving object; and
a camera element connected to the control element and configured to detect a pupil trajectory of the user's pupils corresponding to the moving object,
wherein the control element uses a matching degree between a moving trajectory of the moving object and the pupil trajectory as the feedback signal,
wherein the positive feedback state indicates the matching degree being higher than or equal to a default degree, and the negative feedback state indicates the matching degree being lower than the default degree.

10. The brain stimulation signal control device according to claim 8, further comprising:
a display element connected to the control element and configured to display a test image with a default brightness, wherein the test video presents a moving object; and
a camera element connected to the control element and configured to detect a pupil size of the user's pupils corresponding to the test image,
wherein the control element uses a matching degree between the pupil size and the default brightness as the feedback signal,
wherein the positive feedback state indicates the matching degree being higher than or equal to a default degree, and the negative feedback state indicates the matching degree being lower than the default degree.

11. The brain stimulation signal control device according to claim 8, further comprising:
an input element connected to the control element and configured to receive a user answer on a cognition testing chart, wherein the control element uses a matching degree between the user answer and a default answer as the feedback signal, wherein the positive feedback state indicates the matching degree being higher than or equal to a default degree, and the negative feedback state indicates the matching degree being lower than the default degree.

12. The brain stimulation signal control device according to claim 8, wherein the stimulation parameter comprises a frequency parameter, and the plurality of sensory stimulation signals are capable of arriving at a target area of the user substantially simultaneously, and the plurality of sensory stimulation signals correspond to a same frequency.

13. The brain stimulation signal control device according to claim 8, wherein each one of the plurality of sensory stimulation signals is at least one of a light signal, a sound signal and an electrical signal.

14. The brain stimulation signal control device according to claim 8, wherein the stimulation parameter comprises at least one of a duty ratio parameter and an intensity parameter.

15. A brain stimulation signal control system, comprising:
a brain stimulation signal control device configured to output a plurality of sensory stimulation signals to a user based on a stimulation parameter; and
a mobile device connected to the brain stimulation signal control device, and configured to perform a stimulation feedback procedure to obtain a feedback signal, and set the stimulation parameter as a default parameter of adjust the stimulation parameter according to the feedback signal indicating a positive feedback state or a negative feedback state.

16. The brain stimulation signal control system according to claim 15, wherein the mobile device comprises:
a display element configured to play a test video, wherein the test video presents a moving object;
a camera element configured to detect a pupil trajectory of the user's pupils corresponding to the moving object; and
a computing element connected to the display element and the camera element, and configured to use a matching degree between a moving trajectory of the moving object and the pupil trajectory as the feedback signal, wherein the positive feedback state indicates the matching degree being higher than or equal to a default degree, and the negative feedback state indicates the matching degree being lower than the default degree.

17. The brain stimulation signal control system according to claim 15, wherein the mobile device comprises:
a display element configured to display a test image with a default brightness, wherein the test video presents a moving object;
a camera element configured to detect a pupil size of the user's pupils corresponding to the test image; and
a computing element connected to the display element and the camera element, and configured to use a matching degree between the pupil size and the default brightness as the feedback signal, wherein the positive feedback state indicates the matching degree being higher than or equal to a default degree, and the negative feedback state indicates the matching degree being lower than the default degree.

18. The brain stimulation signal control system according to claim 15, wherein the mobile device comprises:
a display element configured to present a cognition testing chart; and
a computing element connected to the display element, and configured to obtain a user answer on the cognition testing chart, and use a matching degree between the user answer and a default answer as the feedback signal, wherein the positive feedback state indicates the matching degree being higher than or equal to a default degree, and the negative feedback state indicates the matching degree being lower than the default degree.

19. The brain stimulation signal control system according to claim 15, wherein the stimulation parameter comprises a frequency parameter, and the plurality of sensory stimulation signals are capable of arriving at a target area of the user substantially simultaneously, and the plurality of sensory stimulation signals correspond to a same frequency.

20. The brain stimulation signal control system according to claim 15, wherein each one of the plurality of sensory stimulation signals is at least one of a light signal, a sound signal and an electrical signal.

21. The brain stimulation signal control system according to claim 15, wherein the stimulation parameter comprises at least one of a duty ratio parameter and an intensity parameter.
